(19) United States
(12) Patent Application Publication
(10) Pub. No.: US 2016/0369226 A1
(43) Pub. Date: Dec. 22, 2016

(54) SOLID-STATE BIOLOGICAL REACTION DEVICE AND METHOD FOR PREPARING FILAMENTOUS ORGANISM SPORES BY USING THE SAME

(71) Applicant: TIANJIN INSTITUTE OF INDUSTRIAL BIOTECHNOLOGY, CHINESE ACADAMY OF SCIENCE, Tianjin (CN)

(72) Inventors: Wenxia JIANG, Tianjin (CN); Xiaoran ZHANG, Tianjin (CN); Yanhe MA, Tianjin (CN)

(73) Assignee: Tianjin Institute of Industrial Biotechnology, Chinese Acadamy of Sciences, Tianjin (CN)

(21) Appl. No.: 14/901,964

(22) PCT Filed: Dec. 17, 2013
(86) PCT No.: PCT/CN2013/089705

§ 371 Date: Dec. 29, 2015

(30) Foreign Application Priority Data
Nov. 7, 2013 (CN) 201310553403.4

(51) Int. Cl. C12N 3/00; C12N 1/14; C12M 1/12; C12M 1/00; C12M 1/16; C12M 1/06

(52) U.S. Cl. CPC C12N 3/00; C12M 21/16; C12M 27/02; C12M 25/06; C12M 47/02; C12N 1/14

(57) ABSTRACT

A solid-state biological reaction device, comprising a main tank body and a collector is provided. A top air outlet, a bottom air inlet and a material entrance and exit are provided on the main tank body. The collector is connected to the main tank body through the top air outlet. Also provided is a method for preparing filamentous fungus spores utilizing the solid-state biological reaction device, comprising the steps of: feeding a culture substrate into the main tank body through the material entrance and exit, performing steam sterilization by injecting steam through the bottom air inlet, inoculating the filamentous fungus strains onto a sterile culture substrate in the main tank body for culturing so as to obtain mature filamentous fungus spores, and then passing dry air into the main tank body through the bottom air inlet, so that the filamentous fungus spores enter the collector through the top air outlet.

### FIELD OF THE INVENTION

The present invention pertains to the field of biological engineering and specifically, relates to a solid-state biological reaction device and a method for preparing filamentous organism spores by using the same.

### BACKGROUND OF THE INVENTION

In the field of industrial fermentation, enlarged cultivation of strains is an important link of fermentation technology. Only distillery yeast, lactobacillus and a tiny minority of microorganisms have specialized strain production plants, which produce commercial strains directly used in fermentation production. In fermentation enterprises, the process of enlarged cultivation from slant strains to the strains of stage-1 seeding tank is always a technological process with lowest level of mechanization and needs a large amount of manual labor.

Filamentous organisms contain filamentous thallus structures (mycelia), such as: mycelial fungi and actinomycetes. Filamentous organisms are extremely important industrial microorganisms. Most antibiotics are produced through fermentation of actinomycetes. The spores of Beauveria bassiana and Metarrhizium anisopliae are important microbial insecticides. More than 60% of enzyme preparations are produced through fermentation of filamentous organisms. Many organic acids such as: citric acid, gluconic acid and itaconic acid are produced by filamentous organisms. For example, Aspergillus niger is not only used to produce citric acid, itaconic acid and gluconic acid, but also used to produce more than 30 kinds of enzyme preparations. In the Hygienic Standards for Uses of Food Additives GB 2760-2011, more than 26 kinds of enzyme preparations used in foods derive from Aspergillus niger.

Spore inoculation are usually used in cultivation of filamentous organisms, while surface culture (such as: solid culture) for filamentous organisms is generally needed to generate spores.

During submerged fermentation of filamentous organisms, a large amount of strain spores need to be inoculated to a fermentor or a seeding tank. The fermentation of Aspergillus niger needs to use a large amount of spores of Aspergillus niger for inoculation. The Aspergillus niger spores used in industry are mostly bran koji spores prepared through solid fermentation in 1-2 L Erlenmeyer flasks by method of standing and bottle inversion. Following the increase of output and the enlargement of the fermentor, a large amount of bran koji spores will be used in the production. For example, large fermentors of 400-500 m³ are mostly used in fermentation production of citric acid in China at present and 100-200 bottles of bran koji spores cultivated by 1-2 L Erlenmeyer flasks will be needed for each fermentor. In China, more than one million tons of citric acid is produced and more than two million bottles of bran koji spores are used per year.

Preparing bran koji spores in Erlenmeyer flasks cannot realize mechanization and is labor consuming and inefficient. The technology for large-scale preparation of filamentous organism spores has always been a common problem needed to be solved in the fermentation industry.

A large amount of bran koji spores cultivated in Erlenmeyer flasks are used in fermentation production. In addition to the foregoing problem, there are following problems which can hardly be overcome either: firstly, the preparation process of bran koji spores made by Erlenmeyer flask is complex. It is difficult to inspect the quality of bran koji spores flask by flask and extremely liable to be contaminated with contaminating microorganisms, resulting in contamination of the fermentor. Secondly, tens˜hundreds of bottles of bran koji spores need to be inoculated to each fermentor, which is labor consuming and increases the risk of contamination. Thirdly, during inoculation of spores, the bran in the bran koji will enter the fermentor too, increasing the amount of impurities in fermentation broth. It is not suitable to the fermentations which have high requirements for the purity of the fermentation broth. Moreover, in order to prepare plenty of bran koji spores, a large constant-temperature koji-making workshop needs to be built which takes much construction investment.

The VB Spore Box—a device sold by Vogelbusch Biocommodities GmbH and used to produce spores (filamentous microorganism spores in particular) comprises an incubator, a measuring and monitoring section and an air compressor. It is also provided with a sterilizer for agar culture medium and a vacuum collector to form a complete set. In a container similar to glove box, trays are used to cultivate filamentous microorganisms in a solid state. The total usable area of the trays is 7.56 m². The temperature, pressure, humidity and air velocity of the system are controlled. Dry spores are collected in vacuum. From reproduction to harvesting into the bottles, spores are kept in a closed system all the time. This device is sterilized by chemical fumigation. The culture medium needs to be sterilized in another sterilizer before it is transferred aseptically into the Spore Box. Inoculation is conducted aseptically tray by tray in the Spore Box. After the cultivated microorganism spores are mature, pure spores are collected from tray surface under negative pressure. If this Spore Box is used to produce Aspergillus niger spores, about 0.8-1.4 kg spores may be produced in a batch. The production cycle is about 14 days. In other words, five days are for cultivation, five days are for drying and four days are for harvesting, washing, and preparation for the next batch. The structure and configuration of this Spore Box is complex. It spends a long time to wash and make preparation and is labor consuming either. The sterile requirements for equipment, environment and operation are extremely high. In the process of sterilization by chemical fumigation, the transfer of the culture medium from a sterilizer to this device etc., a small mistake may cause microorganism contamination.

After sterilization by chemical fumigation, not only the sterilizing agent must be thoroughly removed to avoid the impact of residual chemical sterilizing agent on cultured microorganism but also the sterilizing agent must be adsorbed and neutralized to prevent the chemical sterilizing agent from polluting the environment. Agar culture medium with high cost is used in solid state cultivation in trays. Spores are collected under negative pressure and the requirements for preventing microorganism contamination of the device are extremely high. Some spores cannot be collected to spore receiving flaks.

### SUMMARY OF THE INVENTION

The object of the present invention is to overcome the defect of existing production equipment for filamentous organism spores, i.e. hardly realizing mechanization and large scale production, and to provide a solid-state biological reaction device, which helps realize mechanized and large-scale production of filamentous organism spores, as well as a method for preparing filamentous organism spores by using this solid-state biological reaction device.

In order to realize the above object, the present invention provides a solid-state biological reaction device, comprising a main tank body and a collector, wherein a top air outlet, a bottom air inlet and a material entrance and exit are provided on the main tank body, and the collector is connected to the main tank body through the top air outlet.

The present invention also provides a method for preparing filamentous organism spores by using this solid-state biological reaction device, including: feeding a culture substrate into the main tank body through the material entrance and exit, performing steam sterilization to the solid-state biological reaction device and culture substrate by injecting steam through the bottom air inlet, inoculating the filamentous organism strains into the main tank body to contact with sterile culture substrate and culturing them so as to obtain mature filamentous organism spores, then passing dry air into the main tank body through the bottom air inlet so that the filamentous organism spores enter into the collector through the top air outlet.

The solid-state biological reaction device of the present invention possessing the following benefits: (1) it can achieve mechanized and large-scale preparation of filamentous organism spores, significantly save human labor and raise production efficiency; (2) it is well sealed and can effectively avoid external contamination to spore cultivation, and when cultivating pathogenic bacteria or bacteria harmful to the environment, it can easily assure environmental safety; (3) the collected spores contain little culture substrate, spores can be maximally collected and minimize wasting; (4) the structure is simple and compact and in-place steam sterilization can be conducted, which is environment-friendly and efficient.

Further, the preparation method and process of filamentous organism spores in the present invention are simple. The entire cultivation process is conducted in a closed system, with low probability of contamination. By controlling the ventilation at the bottom air inlet, the mass collection of spores may be effectively realized. It is particularly conducive to realization of mechanized and large-scale production of filamentous organism spores.

Other features and advantages of the present invention will be described in details in the subsequent embodiments.

### BRIEF DESCRIPTION OF THE DRAWINGS

The accompanying drawings are intended to provide further understanding on the present invention, which constitute a part of the description and explain the present invention together with the following embodiment and make no limitation for the present invention. In the drawings:

FIG. 1 is a schematic of a solid-state biological reaction device according to a preferred embodiment of the present invention.

FIG. 2 is a schematic of the spatial position of the support balls and culture substrate during the cultivation process according to a preferred embodiment of the present invention.

### DETAILED DESCRIPTION OF THE EMBODIMENTS

Below the embodiments of the present invention are described. It should be understood that the embodiments described here are intended to illustrate and not to limit the present invention.

Below the solid-state biological reaction device of the present invention is described by taking preparing filamentous organism spores by using the solid-state biological reaction device of the present invention for example, but this does not mean the solid-state biological reaction device of the present invention is only limited to prepare filamentous organism spores.

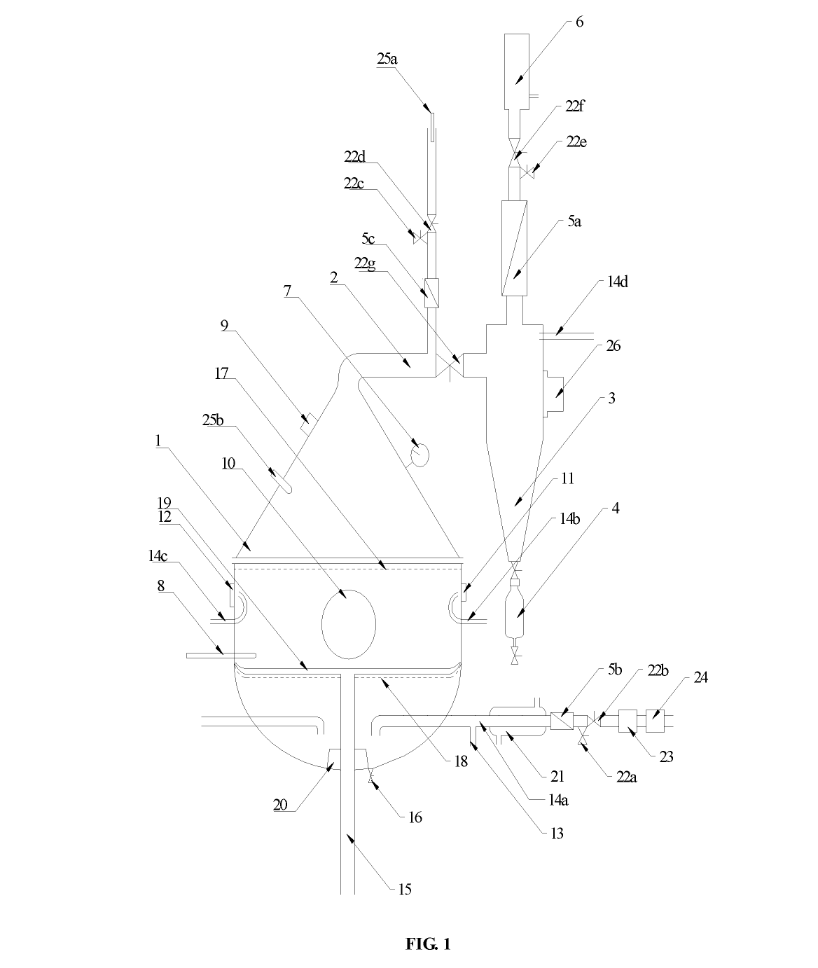

As shown in FIG. 1, the present invention provides a solid-state biological reaction device, comprising a main tank body 1 and a collector 3, wherein a top air outlet, a bottom air inlet and a material entrance and exit 10 are provided on the main tank body 1 and the collector 3 is connected to the main tank body 1 through the top air outlet.

In the present invention, the top air outlet may be connected to the main tank body 1 through an exhaust pipe 2. Preferably, exhaust pipe 2 is a 3-way pipe. One end of the 3-way pipe is connected to the main tank body 1, another end is connected to the collector 3 and another end is connected to a filter 5c. The end connecting the filter may be used as the exhaust pipeline of the main tank body 1 and can not only prevent external microorganisms from contaminating the material in the main tank body 1 but also prevent the material in the main tank body 1 (e.g. filamentous organism spores) from entering external environment through the top air outlet, thus pollute environment or do harm to the health of operators. A valve may be disposed at each end of the 3-way pipe, for example: valve 22c, valve 22d and valve 22g. Further, in order to detect the humidity of the gas discharged from the main tank body 1, a thermohygrograph probe 25a may be disposed at the end of the exhaust pipeline. A thermohygrograph probe 25b may be directly disposed on the main tank body 1 to detect the humidity inside the main tank body 1 more accurately.

In the present invention, in the main tank body 1, a screen 17 may further be disposed, under the top air outlet, to restrain the material with an average particle size above 1 mm in the main tank body 1 from passing the top air outlet. The screen 17 may effectively reduce the amount of culture substrate entering the collector 3 together with filamentous organism spores.

Further, in the main tank body 1, a support orifice plate 18 may further be disposed. It is a perforated support plate, which can support the material in the main tank body 1 and allow gas to pass the bottom air inlet and the support orifice plate 18 successively and then to contact with the material. The support orifice plate 18 may increase air inflow area, thereby benefiting to steam sterilization (thorough sterilization) and aeration during cultivation (sufficient contact between the material and air).

Further, in the main tank body 1, an agitating structure may further be disposed. The agitating structure comprises a stirring paddle 19 and a stirring shaft 15. Preferably, the stirring shaft 15 is connected to the main tank body 1 through a shaft seal 20. Further, the stirring shaft may be connected to a motor, thereby regulating and controlling the agitating structure in a better way and adapting to different demands. The present invention does not have particular limitation to the setting mode of the agitating structure. In other words, the agitating structure may be any common agitating structures.

According to one preferred embodiment of the present invention, in the main tank body 1, a screen 17, a support orifice plate 18 and an agitating structure are further disposed. Further, a tank bottom valve 16 may be disposed at the bottom of the main tank body 1 to facilitate steam sterilization and water discharge.

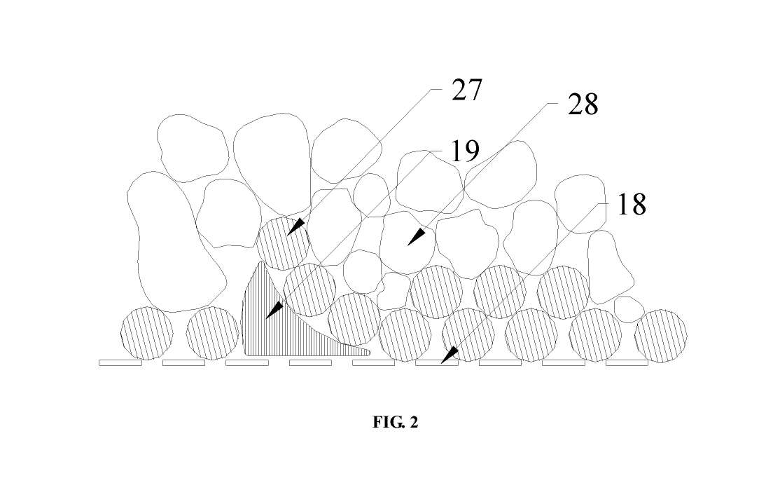

In the present invention, preferably, the solid-state biological reaction device comprises a support orifice plate 18 and a plurality of support balls 27 (refer to FIG. 2). The use of support balls can effectively prevent the material in the main tank body 1 from blocking the support orifice plate and/or maximally avoid the stirring paddle damaging the material. In order that the support balls effectively play the foregoing role, those skilled in the art can easily select the size and shape of the holes on the support orifice plate and the size and shape of the support balls. For example, if the support balls are in a spherical shape, it is preferred to set the holes on the support orifice plate in a non-round shape, such as one or more of polygon (such as: triangle), ellipse and irregular shape.

More preferably, the quantity of the support balls 27 enables the support balls to cover at least the whole support orifice plate 18, thereby separating the material from the support orifice plate 18 in the main tank body 1. As shown in FIG. 2, in actual use, the support balls 27 are placed on the support orifice plate 18 and can separate the material 28 from the support orifice plate 18 in the main tank body 1, but they won't affect the gas from passing the support orifice plate and contacting with the material in the main tank body 1, so the existence of the support balls can effectively prevent the material in the main tank body 1 from blocking the support orifice plate 18, thereby particularly improving aeration. Moreover, the existence of the support balls 27 can impede direct contact between the stirring paddle 19 and the material 28 in the main tank body 1, so it can effectively weaken the destruction of the material by the stirring paddle during stirring. Particularly during preparation of filamentous organism spores, this setting can avoid damage of mycelia and successively prevent the output of filamentous organism spores from being affected.

Still more preferably, the density of the support balls is greater than that of the material in the main tank body, thereby prompting the support balls to directly contact with the support orifice plate in the whole cultivation process and give a better play to their role in preventing the material from blocking the support orifice plate. There are no special requirements for the material of the support balls as long as it can endure the temperature and pressure of steam sterilization. Moreover, they can be hollow or solid.

In the present invention, there may be one or more bottom air inlets on the main tank body 1. When there is one bottom air inlet, steam may be input from this bottom air inlet to realize sterilization at first and after inoculation, sterile air is input from this bottom air inlet for aeration. Considering the convenience of operation, preferably the bottom air inlet of the main tank body 1 is connected to one end of a 3-way pipe and other two ends of the 3-way pipe are connected to a steam pipe 13 and an air pipe 14a respectively. In order to sterilize more thoroughly, a steam pipe may be connected to the collector 3. In order to keep the collector 3 dry, an air pipe 14d may further be connected to the collector 3.

Wherein, the air pipe 14a may be connected to a heat exchanger 21 and a humidifier 23 so that the temperature and humidity of the air reach specific values before entering into the main tank body 1, thereby adjusting and controlling the temperature and humidity of the material in the main tank body 1. In order to control the temperature in the main tank body 1 more stably, a jacket or coil may be disposed outside the main tank body 1 in a way known to those skilled in the art. The air pipe 14a may further be connected to a filter 5b, to remove microorganisms in the air. The air pipe 14a may further be connected to an air flow controller 24, to control the flow of the air entering into the main tank body 1 more conveniently.

In the present invention, the collector 3 may further be connected to a filter 5a and a vacuum generator 6 successively. The start of the vacuum generator can promote filamentous organism spores to enter into the collector 3. In order to make for sterilization, a valve 22e and a valve 22f may be disposed between the filter 5a and the vacuum generator 6.

In the present invention, the collector 3 may be any type of closed container. Preferably, the collector 3 is a cyclone separator. The material inlet on the cyclone separator is connected to the top air outlet on the main tank body 1 through the exhaust pipe 2. More preferably, the top outlet of the cyclone separator is connected to a filter 5a and a vacuum generator 6 successively. There are not special requirements for the concrete use method of the cyclone separator and those skilled in the art can make choice according to the properties of the material entering the collector. Here no unnecessary details are given.

In order to collect the filamentous organism spores collected in the cyclone separator more conveniently, preferably, a detachable collection bottle 4 is connected to the bottom of the cyclone separator. In order to recover the spores entering the cyclone separator more sufficiently, more preferably, a vibrator 26 is provided on the main body of the cyclone separator. By starting the vibrator, some of the filamentous organism spores adhering to the wall of the cyclone separator and the filter 5a may be prompted to enter the collection bottle 4.

In the present invention, in order to get the pressure and temperature in the main tank body 1 more conveniently, a pressure gauge 7 and a thermometer 8 may be disposed on the main tank body 1. In order to inoculate filamentous organism strains into the main tank body 1 more conveniently, an inoculation inlet 9 may be disposed on the main tank body 1. In order to easily observe the growth condition of filamentous organisms and the generation condition of spores in the main tank body 1, a lamp hole 11 and a sight glass 12 may be disposed on the main tank body 1. In actual operation, the lamp hole 11 and the sight glass 12 are liable to be covered by the material. Therefore, for easy observation, air pipes 14b and 14c may be connected to the lamp hole 11 and the sight glass 12 respectively. If needed, sterile air is input to blow away the material adhering to the lamp hole and the sight glass and then observe the culture condition in the main tank body 1.

Further, in order to carry out steam sterilization and sterile operation and make for control of every connecting unit, a plurality of valves may be disposed. Those skilled in the art can easily set the valves, for example, valve 22a and valve 22b provided on the air pipe 14a; valve 22c, valve 22d and valve 22g provided on the exhaust pipe 2; valve 22e and valve 22f provided between the filter 5a and vacuum generator 6. No unnecessary details are given here.

The present invention also provides a method for preparing filamentous organism spores by using the foregoing solid-state biological reaction device of the present invention. The solid-state biological reaction device comprises a main tank body 1 and a collector 3, wherein a top air outlet, a bottom air inlet and a material entrance and exit 10 are provided on the main tank body, and the collector 3 is connected to the main tank body 1 through the top air outlet. The method includes the following steps: feeding a culture substrate into the main tank body 1 through the material entrance and exit, performing steam sterilization to the solid-state biological reaction device and culture substrate by injecting steam through the bottom air inlet, inoculating the filamentous organism strains into the main tank body 1 to contact with sterile culture substrate and culturing them so as to obtain mature filamentous organism spores, then passing dry air into the main tank body 1 through the bottom air inlet so that the filamentous organism spores enter into the collector 3 through the top air outlet.

In the present invention, the top air outlet may be connected to the main tank body 1 through an exhaust pipe 2. Preferably, exhaust pipe 2 is a 3-way pipe. One end of the 3-way pipe is connected to the main tank body 1, another end is connected to the collector 3 and another end is connected to a filter 5c. The end connecting the filter may be used as an exhaust pipeline of the main tank body 1 and can not only prevent external microorganisms from contaminating the material in the main tank body 1 but also prevent the material in the main tank body 1 (mainly filamentous organism spores) from entering external environment through the top air outlet, thus pollute environment or do harm to the health of operators. A valve may be disposed at each end of the 3-way pipe, for example: valve 22c, valve 22d and valve 22g. Further, in order to detect the humidity of the gas discharged from the main tank body 1, a thermohygrograph probe 25a may be disposed at the end of the exhaust pipeline.

A thermohygrograph probe 25b may be directly disposed on the main tank body 1 to detect the humidity inside the main tank body 1 more accurately.

In the present invention, in the main tank body 1, a screen 17 may further be disposed, under the top air outlet, to restrain the material with an average particle size above 1 mm (other than the filamentous organism spores) in the main tank body 1 from passing the top air outlet. The screen 17 may effectively reduce the amount of culture substrate entering the collector 3 together with filamentous organism spores.

Further, in the main tank body 1, a support orifice plate 18 may further be disposed. It is a perforated support plate, which can support the material in the main tank body 1 and allow gas to pass the bottom air inlet and the support orifice plate 18 successively and then to contact with the material. Culture substrates (or materials) are placed on the support orifice plate 18. The support orifice plate 18 may increase air inflow area, thereby benefiting to steam sterilization (thorough sterilization) and aeration during cultivation (sufficient contact between the material and air).

Further, in the main tank body 1, an agitating structure may further be disposed. The agitating structure comprises a stirring paddle 19 and a stirring shaft 15. During inoculation, the agitating structure may be started to mix filamentous organism strains and culture substrate even better. During cultivation, the agitating structure may be started to stir the material (usually, an intermittent stirring mode may be adopt during the cultivation of filamentous organisms, to prevent massive damage of mycelia), thereby promoting aeration and preventing agglomeration of the material. Preferably, the stirring shaft 15 is connected to the main tank body 1 through a shaft seal 20. Further, the stirring shaft may be connected to a motor, thereby regulating and controlling the agitating structure in a better way and adapting to different demands for preparing filamentous organism spores. The present invention does not have particular limitation to the setting mode of the agitating structure. In other words, the agitating structure may be any common agitating structures.

According to one preferred embodiment of the present invention, in the main tank body 1, a screen 17, a support orifice plate 18 and an agitating structure are further disposed. Further, a tank bottom valve 16 may be disposed at the bottom of the main tank body 1 to facilitate steam sterilization and water discharge.

In the present invention, preferably, the solid-state biological reaction device comprises a support orifice plate 18 and a plurality of support balls 27 (refer to FIG. 2). The use of support balls can effectively prevent the material in the main tank body 1 from blocking the support orifice plate and/or maximally avoid the stirring paddle damaging the material. In order that the support balls effectively play the foregoing role, those skilled in the art can easily select the size and shape of the holes on the support orifice plate and the size and shape of the support balls. For example, if the support balls are in a spherical shape, it is preferred to set the holes on the support orifice plate in a non-round shape, such as one or more of polygon (such as: triangle), ellipse and irregular shape.

More preferably, the quantity of the support balls 27 enables the support balls to cover at least the whole support orifice plate 18, thereby separating the material from the support orifice plate 18 in the main tank body 1. As shown in FIG. 2, the support balls 27 are placed on the support orifice plate 18 (i.e.: the support balls are sent into the main tank body 1 before the culture substrate) and can separate the material 28 from the support orifice plate 18 in the main tank body 1, but they won't affect the gas from passing the support orifice plate and contacting with the material in the main tank body 1, so the existence of the support balls can effectively prevent the material in the main tank body 1 from blocking the support orifice plate 18, thereby particularly improving aeration. Moreover, the existence of the support balls 27 can impede direct contact between the stirring paddle 19 and the material 28 in the main tank body 1, so it can effectively weaken the destruction of the material by the stirring paddle during stirring. This setting can avoid damage of mycelia and successively prevent the output of filamentous organism spores from being affected.

Still more preferably, the density of the support balls is greater than that of the material in the main tank body, thereby prompting the support balls to directly contact with the support orifice plate in the whole cultivation process and give a better play to their role in preventing the material from blocking the support orifice plate. There are no special requirements for the material of the support balls as long as it can endure the temperature and pressure of steam sterilization. Moreover, they can be hollow or solid.

In the present invention, there may be one or more bottom air inlets on the main tank body 1. When there is one bottom air inlet, firstly steam may be input from this bottom air inlet to realize sterilization at first and after inoculation, sterile air is input from this bottom air inlet. Considering the convenience of operation, preferably the bottom air inlet of the main tank body 1 is connected to one end of a 3-way pipe and other two ends of the 3-way pipe are connected to a steam pipe 13 and an air pipe 14a respectively. Steam is input to the solid-state biological reaction device through the steam pipe 13 for steam sterilization. Sterile air is input to the main tank body 1 through the air pipe 14a. In order to sterilize more thoroughly, a steam pipe may be connected to the collector 3. In order to keep the collector 3 dry, an air pipe 14d may further be connected to the collector 3.

Wherein, the air pipe 14a may be connected to a heat exchanger 21 and a humidifier 23 so that the temperature and humidity of the air reach specific values before entering into the main tank body 1, thereby adjusting and controlling the temperature and humidity of the material in the main tank body 1. In order to control the temperature in the main tank body 1 more stably, a jacket or coil may be disposed outside the main tank body 1 in a way known to those skilled in the art. The air pipe 14a may further be connected to a filter 5b, to remove microorganisms in the air. The air pipe 14a may further be connected to an air flow controller 24, to control the flow of the air entering into the main tank body 1 more conveniently.

In the present invention, the collector 3 may further be connected to a filter 5a and a vacuum generator 6 successively. When dry air is input to the main tank body 1, the vacuum generator 6 is started to promote filamentous organism spores to enter into the collector 3. In order to make for sterilization, a valve 22e and a valve 22f may be disposed between the filter 5a and the vacuum generator 6.

In the present invention, the collector 3 may be any type of closed container. Preferably, the collector 3 is a cyclone separator. The material inlet on the cyclone separator is connected to the top air outlet on the main tank body 1 through the exhaust pipe 2. More preferably, the top outlet of the cyclone separator is connected to the filter 5a and the vacuum generator 6 successively. There are not special requirements for the concrete use method of the cyclone separator and those skilled in the art can make choice according to the properties of the material entering the collector. Here no unnecessary details are given.

In order to collect the filamentous organism spores collected in the cyclone separator more conveniently, preferably, a detachable collection bottle 4 is connected to the bottom of the cyclone separator. In order to recover the spores entering the cyclone separator more sufficiently, more preferably, a vibrator 26 is provided on the main body of the cyclone separator. By starting the vibrator, some of the filamentous organism spores adhering to the wall of the cyclone separator and the filter 5a may be prompted to enter the collection bottle 4.

In the present invention, in order to get the pressure and temperature in the main tank body 1 more conveniently, a pressure gauge 7 and a thermometer 8 may be disposed on the main tank body 1. In order to inoculate filamentous organism strains into the main tank body 1 more conveniently, an inoculation inlet 9 may be disposed on the main tank body 1. In order to easily observe the growth condition of filamentous organisms and the generation condition of spores in the main tank body 1, a lamp hole 11 and a sight glass 12 may be disposed on the main tank body 1. During cultivation, the lamp hole 11 and the sight glass 12 are liable to be covered by the material. Therefore, for easy observation, air pipes 14b and 14c may be connected to the lamp hole 11 and the sight glass 12 respectively. If needed, sterile air is input to blow away the material adhering to the lamp hole and the sight glass and then observe of the culture condition in the main tank body 1.

Further, in order to carry out steam sterilization and sterile operation and make for control of every connecting unit, a plurality of valves may be disposed. Those skilled in the art can easily set the valves, for example, valve 22a and valve 22b provided on the air pipe 14a; valve 22c, valve 22d and valve 22g provided on the exhaust pipe 2; valve 22e and valve 22f provided between the filter 5a and the vacuum generator 6. No unnecessary details are given here.

In the present invention, to drive filamentous organism spores to enter the collector 3, those skilled in the art can select the flow of dry air, but preferably, relative to a main tank body with a volume of 300 L, the flow of dry air input to the main tank body 1 should be 0.1-600 L/min. Wherein the dry air in the present invention refers to the air basically containing no water, for example, the air with a dew point below 20° C. (dew point is the condensation temperature of water at 0.101 MPa, a lower dew point indicates less moisture present in the air.). In order to promote filamentous organism spores to enter the collector 3. When dry air is input to the main tank body 1, the agitating structure may be started and stir at a high speed.

In the present invention, according to the type of the microorganisms to be cultivated, those skilled in the art can easily determine the formula and dose of the culture substrate and the culture conditions (temperature, humidity, aeration rate and time) etc., so no unnecessary details are given here.

In the method of the present invention for preparing filamentous organism spores by using the solid-state biological reaction device, the culture substrate is particulate matter which filamentous organism can adhere to and provide nutrition for the growth of filamentous organism. It may be any of the culture substrates commonly used in the art. Preferably, the average particle size of the culture substrate is 4-40 mm (more preferably, 15-20 mm) and the culture substrate is at least one of corncob, straw and cane trash. Preferably, the culture substrate is pulverized corncob with an average particle size of 15-20 mm. It should be noted that in order to provide more suitable nutrients, carbon source and/or nitrogen source (such as: bran extract liquid, glucose or (NH₄)₂SO₄) may be added to the main tank body 1 before steam sterilization. The a carbon source and/or nitrogen source may be added to the main tank body 1 together with the culture substrate, or may be added to the main tank body 1 in a time different from the addition of the culture substrate. In order to distribute them more evenly and obtain a better cultivation effect, preferably, the culture substrate is soaked in a water solution containing carbon source and/or nitrogen source (such as: bran extract liquid) for 0.1-24 h before inputting into the main tank body 1.

Below the present invention will be described in details by referring to examples. In the following examples, the filamentous organism strains are Aspergillus niger (strain No.: ATCC 10864; inoculation amount: 30 spores/g of culture substrate). Composition of the culture substrate (10 kg): 5 kg of granulate corncob (the weight of the corncob with an average particle size above 5 mm accounts for 95% of the total weight) and 5 kg of bran extract liquid, pH 5.5-6.0. Method for preparing the bran extract liquid: mix bran and water at a weight ratio of 1:20, heat the mixture to boiling and then filter out particulate matter by cotton cloth, the filtrate is adopted as the bran extract liquid.

### EXAMPLE 1

Preparing filamentous organism spores by using the solid-state biological reaction device provided in an embodiment of the present invention.

The used solid-state biological reaction device comprises a main tank body 1 (300 L) and a cyclone separator 3, wherein a top air outlet, a bottom air inlet and a material entrance and exit are provided on the main tank body 1 and the material inlet on the cyclone separator is connected to the top air outlet on the main tank body 1 through an exhaust pipe 2 (a 3-way pipe: one end is connected to the main tank body 1, one end is connected to the cyclone separator 3 and another end is connected to a filter 5c), while the top outlet of the cyclone separator is connected to a filter 5a and a vacuum generator 6 successively, the bottom of the cyclone separator is connected to a detachable collection bottle 4 and a vibrator 26 is provided on the main body of the cyclone separator; A screen 17, a support orifice plate 18 and an agitating structure are further disposed in the main tank body 1. The screen 17 (pore diameter: 1 mm) is placed under the top air outlet and used to restrain the material with an average particle size above 1 mm in the main tank body 1 from passing the top air outlet. The support orifice plate 18 is a perforated support plate, which can support the material in the main tank body 1 and allow gas to pass the bottom air inlet and the support orifice plate 18 successively and then to contact with the material (the orifices on it are equilateral triangle with a side length of 1 cm). The agitating structure is a structure comprising a stirring paddle 19 and a stirring shaft 15 (the spacing between the stirring paddle 19 and the support orifice plate 18 is 1 mm, and the stirring shaft 15 is connected to a motor); the bottom air inlet of the main tank body 1 is connected to one end of a 3-way pipe and other two ends of the 3-way pipe are connected to a steam pipe 13 and an air pipe 14a respectively, wherein the air pipe 14a connects a heat exchanger 21, a humidifier 23, a filter 5b and an air flow controller 24; on the main tank body 1, a pressure gauge 7, a thermometer 8, an inoculation inlet 9, a thermohygrograph probe 25b, a lamp hole 11 and a sight glass 12 are further disposed; the solid-state biological reaction device also comprises a plurality of support balls 27 (solid ceramic balls with a density of 3.6 g/cm³) with a diameter of 2 cm, and the support balls are on the support orifice plate 18 and their quantity enables the support balls to cover the stirring paddle; further, the solid-state biological reaction device also comprises valve 22a and valve 22b provided on the air pipe, valve 22c, valve 22d and valve 22g provided on the exhaust pipe 2, and valve 22e and valve 22f provided between the filter 5a and the vacuum generator 6.

During preparing filamentous organism spores, culture substrate (the feed amount is 10 kg) is fed to the main tank body 1 through the material entrance and exit and placed on the support balls. Steam is input from the steam pipe 13 to perform steam sterilization (121° C., 0.1 MPa, 40 min) to the solid-state biological reaction device and the culture substrate. When the temperature of the culture substrate is reduced to 37° C., filamentous organism strains are inoculated to the main tank body 1 through the inoculation inlet 9 under stirring (stirring speed: 10 r/min) and is mixed with sterile culture substrate. 20 min later, the stirring is stopped and air is input to cultivate the filamentous organism strains. The air flow is controlled at 0.5 L/min to obtain mature filamentous organism spores. 5 days later, dry air (dew point: 10° C.) is input to the main tank body 1 from the air pipe 14a. The flow of the dry air is controlled at 0.5 L/min. Two days later, air flow is raised to 100 L/min, the stirring speed is controlled at 20 r/min and the vacuum generator 6 is started so that filamentous organism spores enter the cyclone separator 3 through the exhaust pipe 2. After cyclone separation, filamentous organism spores enter the collection bottle 4. Lastly, the vibrator 26 is started so that some of the filamentous organism spores adhering to the wall of the cyclone separator and the filter 5a enter the collection bottle 4 and eventually 0.8 kg of filamentous organism spores are obtained.

When air is input to cultivate filamentous organism strains, the agitating structure is started to intermittently stir the material, the stirring speed is controlled at 3 r/min, and the stirring is performed for 2 min every 12 h; the pressure and temperature in the main tank body 1 are acknowledged from the pressure gauge 7 and thermometer 8 and the humidity in the main tank body is acknowledged from the thermohygrograph probe 25b, in order to adjust the humidity and temperature of input air (control the temperature in the main tank body 1 at 37° C., pressure at 0.02-0.1 MPa and humidity at 70-100%). The operators may obtain the growth condition of filamentous organism and the generation condition of spores in the main tank body 1 from the lamp hole 11 and sight glass 12.

The foregoing example indicates the method provided in the present invention can realize mechanized and large-scale preparation of filamentous organism spores, and significantly save human labor and has high production efficiency.

Above preferable embodiments of the present invention are described in details, but the present invention is not limited to the concrete details of the embodiment. Within the scope of technical thinking of the present invention, the technical scheme of the present invention may be modified in a simple way. These simple modifications all are within the protective scope of the present invention.

Further, it should be noted that the technical features described in the foregoing embodiment may be combined in any appropriate way as long as no conflict is aroused. In order to avoid unnecessary repetition, the present invention does not describe all the possible combinations.

Further, the embodiments of the present invention may also be freely combined. As long as they are not against the principle of the present invention, they shall also be deemed as the content disclosed by the present invention.

1. A solid-state biological reaction device, comprising a main tank body and a collector, wherein a top air outlet, a bottom air inlet and a material entrance and exit are provided on the main tank body and the collector is connected to the main tank body through the top air outlet; a support orifice plate and an agitating structure is further provided in the main tank body, the support orifice plate is a perforated support plate, which can support the material in the main tank body and allow gas to pass the bottom air inlet and the support orifice plate successively and then to contact with the material; the agitating structure is a structure comprising a stirring paddle and a stirring shaft; the solid-state biological reaction device comprises a support orifice plate and a plurality of support balls, the quantity of support balls enable the support balls to cover at least the whole support orifice plate, thereby separating the material from the support orifice plate in the main tank body.

2. The solid-state biological reaction device according to claim 1, wherein at least one of a screen, a support orifice plate and an agitating structure is further provided in the main tank body, the screen is under the top air outlet and used to restrain the material with an average particle size above 1 mm in the main tank body from passing the top air outlet; the support orifice plate is a perforated support plate, which can support the material in the main tank body and allow gas to pass the bottom air inlet and the support orifice plate successively and then to contact with the material; the agitating structure is a structure comprising a stirring paddle and a stirring shaft; preferably, a screen, a support orifice plate and an agitating structure are further provided in the main tank body.

3. The solid-state biological reaction device according to claim 12, wherein the solid-state biological reaction device comprises a support orifice plate and a plurality of support balls, the quantity of support balls enable the support balls to cover at least the whole support orifice plate, thereby separating the material from the support orifice plate in the main tank body; preferably, the density of the support balls is higher than that of the material in the main tank body.

4. The solid-state biological reaction device according to claim 1, wherein the bottom air inlet of the main tank body is connected to one end of a 3-way pipe and other two ends of the 3-way pipe are connected to a steam pipe and an air pipe respectively.

5. The solid-state biological reaction device according to claim 1, wherein the collector is further connected to a filter and a vacuum generator successively.

6. The solid-state biological reaction device according to claim 5, wherein the collector is a cyclone separator and the material inlet on the cyclone separator is connected to the top air outlet on the main tank body through an exhaust pipe; the top outlet of the cyclone separator is connected to the filter and vacuum generator successively.

7. The solid-state biological reaction device according to claim 6, wherein a detachable collection bottle is connected to the bottom of the cyclone separator, a vibrator is provided on the main body of the cyclone separator.

8. A method for preparing filamentous organism spores by using the solid-state biological reaction device according to claim 1;
the method includes the following steps: feeding a culture substrate into the main tank body through the material entrance and exit, performing steam sterilization to the solid-state biological reaction device and culture substrate by injecting steam through the bottom air inlet, inoculating the filamentous organism strains into the main tank body to contact with sterile culture substrate and culturing them so as to obtain mature filamentous organism spores, then passing dry air into the main tank body through the bottom air inlet so that the filamentous organism spores enter into the collector through the top air outlet.

9. The method according to claim 8, wherein the bottom air inlet of the main tank body is connected to one end of a 3-way pipe and other two ends of the 3-way pipe are connected to a steam pipe and an air pipe respectively, steam is input to the solid-state biological reaction device from the steam pipe to perform steam sterilization, and sterile air is input to the main tank body from the air pipe.

10. The method according to claim 8, wherein the collector is further connected to a filter and a vacuum generator successively; when dry air is input to the main tank body, the vacuum generator is started to prompt filamentous organism spores to enter into the collector.

11. The method according to claim 8, wherein the average particle size of the culture substrate is 4-40 mm, and the culture substrate is at least one of corncob, straw and cane trash.

12. The method according to claim 11, wherein the average particle size of the culture substrate is 15-20 mm.

13. The method according to claim 8, wherein the culture substrate is pulverized corncob with an average particle size of 15-20 mm.
